(19) United States
(12) Patent Application Publication
(10) Pub. No.: US 2017/0013792 A1
(43) Pub. Date: Jan. 19, 2017

(54) Micro-Biologically Favoring Fungus Based Organic Biocomposite Substrate Having Superior Capillary Dynamics

(71) Applicant: Hans Steve Croteau, San Diego, CA (US)

(72) Inventor: Hans Steve Croteau, San Diego, CA (US)

(21) Appl. No.: 15/202,588

(22) Filed: Jul. 6, 2016

Related U.S. Application Data
(60) Provisional application No. 62/192,567, filed on Jul. 15, 2015.
(60) Provisional application No. 62/298,473, filed on Feb. 23, 2016.

(51) Int. Cl. A01G 31/00; A01G 9/10; A01G 1/04; C12N 1/14; C12N 11/00

(52) U.S. Cl. CPC A01G 31/001; C12N 1/14; C12N 11/00; A01G 1/04; A01G 9/1073

(57) ABSTRACT

A soil-less biodegradable and porous rigid foam substrate for cultivating plants that has been formed of a heterogeneous matrix of organic fibers pasteurized with organic matter and natural minerals, and then contacted with a fungus, resulting in a biocomposite foam with excellent capillary dynamics, the whole of which does not compact and whose organic bio-composition favors the proper micro-biological activity upon a plants' rhizosphere. The preferred embodiment is to grow said substrate inside a waxed hexagonal prism shaped box, and around an externally accessible means of internal aeration, for approximately 3-5 days, and then to deactivate the live fungus in said biocomposite foam by exposure to microwave radiation.

### CROSS REFERENCE TO RELATED APPLICATIONS

This application claims priority based on provisional application Ser. No. 62/192,567, filed Jul. 15, 2015, and provisional application Ser. No. 62/298,473, filed on Feb. 23, 2016, both contents of which are incorporated herein by reference in their entirety.

### FIELD OF THE INVENTION

This invention relates to the biology(1), organic(2) chemistry(3), structure, and composition of soilless plant cultivation substrates made of one part fibers and carbohydrates of organic matter(4), and whose remaining part is limited to natural Earth minerals(5), with the resulting substrate's innate biochemistry (compounds), structure, and composition being beneficial to its guest plant in several ways, but not as an essential macronutrient, micronutrient, or trace element (fertilizers); this invention also relates to an internal means of increasing the dissolved oxygen in the nutrient solution of said substrate, and all methods of making said substrates, inclusive of the inherent means of delivering said compounds to a plants' rhizosphere.

See: (1) https://en.wikipedia.org/wiki/Biology,

(2) https://www.ams.usda.gov/grades-standards/organic-standards,
(3) https://en.wikipedia.org/wiki/Organic_chemistry, Not limited to inanimate matter; organic matter defines live or dead (inactive) mushroom mycelium. Also see:
(4) https://en.wikipedia.org/wiki/Organic_matter, https://en.wikipedia.org/wiki/Mineral, and
(5) https://en.wikipedia.org/wiki/Mineral. (The contents of websites (1) through (5), footnoted above, are adopted in their entirety as though fully set forth at length herein.)

### BACKGROUND OF THE INVENTION

“This invention relates to plant containers [and substrates] capable of growing plants by hydroponic means. Hydroponics is a method of growing plants using an inert growing medium saturated with plant nutrient solution, where soil is not required. The plant's roots attach to or root in the inert growing medium and the plant uses the growing medium as it would use soil, to absorb nutrients required by the plant to grow and for mechanical support to hold the plant upright.” [amendment added.] (See U.S. Pat. No. 8,667,734, Granted on Mar. 11, 2014, to Johnson et al.)

“Root substrates (substrates) are commonly used in the production of containerized greenhouse and nursery crops (Bunt 1988; Nelson, 2003). Substrates are formulated from various inorganic and organic components to provide suitable physical and chemical properties as required by the specific crop and growing conditions (Bunt, 1988).” (See Root Substrate pH, Electrical Conductivity, and Macroelement Concentration of Sphagnum Peat-based Substrates Amended with Parboiled Fresh Rice Hulls or Perlite; Mary M. Gachukia and Michael R. Evans, HortTechnology, October-December 2008, vol. 18, no. 4, pp. 644-649.)

There is no shortage of prior art concerning soilless growing mediums. “A wide variety of potting soils and other growing medium have been developed for growing potted plants. Perhaps the most common are mixtures having a base of sphagnum peat moss, soil, ground coconut coir, composted hardwood bark, or composted manure. Soilless potting mixes are desirable because quality top soil . . . can be inconsistent in terms of air and water circulation and can also contain diseased organisms. Achieving the best growing results requires balance in terms of chemistry, composition and structure of the soil or the cultivation medium.” (See U.S. Pat. No. 8,911,525 B1, Pub. on Dec. 16, 2014, to Nano Growth Technologies.)

There are several direct biochemical, structural, and mechanical factors that are crucial to the performance of a substrate and that are not plant fertilizers. These include, but are not limited to, PH (Potentia Hydrogenii, or Potential Hydrogen), PPM (Parts Per Million) of any and all chemical elements on the periodic table and any and all of their molecular configurations, Temperature, Biocompatibility, Density, Cationic Exchange Capacity, Electrical Conductivity, Percentage of Air Filled Pores, Permeability (Gas Exchange Rate), and of course, Absorbability.

There are several indirect factors that are also not plant fertilizers, and which are nevertheless desired qualities of a substrate. These include, but are not limited to, that the substrate be free from Pathogens and Pests, and yet be Inexpensive, Evaporation Resistant, Compaction Resistant, Biodegradable, Compostable, and Whose Biocomposition of Renewable Organic Fibers and Natural Resources Favor the Proper Aerobic Micro-Biological(6) Activity. (For footnote(6), see Vallance, D'eniel, Floch, et al., Pathogenic and beneficial microorganisms in soilless cultures; Agronomy for Sustainable Development, January 2011, Sec. 2.1., Influence of the kind of substrate on microflora.)

“Plant growth media are described as composed of various combinations of vermiculite, perlite, and clay and having superior properties for use as potting soil for container-grown plants. Such media are characterized by greater moisture capillarity and available moisture retention. There is also less danger of plant injury through nutrient deficiencies, over-fertilization, or pH changes. Such media are also characterized by their dry state physical properties in the form of non-compacting, light weight, non-vitreous, rigid foam structures. [T]he media-containers can be inverted without dislodging their contents and the media can be repeatedly wetted and dried while maintaining the desired foam structure . . . Clay has been used since the dawn of history as a binder to form bricks and the like, but . . . [except for its high cation exchange capacity(7), clay] has never been regarded as a desirable medium for growing plants.” [amendment and emphasis added.] (For fn(7), See U.S. Pat. No. 4,168,962 A, Pub. on Sep. 25, 1979, to Victor M. Lambeth; See US Patent No. US 20,040,111,968 A1, Pub. on Jun. 17, 2004, to Danny Day entitled “Production and use of a soil amendment . . . ” and at ¶14 it is disclosed that “Charcoal [also known as “Biochar”] also acts to increase soil's water holding capacity and increase cation exchange capacity. (Glaser, 1999)).” [a.k.a added.]

“[Soilless] [h]orticultural growing media are currently available in a variety of forms. Media may be produced from natural or synthetic materials. Some growing media are made from loose materials, such as peat and vermiculite. Other growth media are shaped, usually composed of phenolic foam, bonded foam, bonded peat, or wrapped peat, or a fibrous material, such as rock wool. Shaped growing materials are [generally] stabilized and held together by incorporation of a synthetic adhesive.” [amendment and emphasis added.] (See U.S. Pat. No. 8,671,616, Pub. on Mar. 18, 2014, to Kennedy et al.) Foam media made entirely from organic rice hulls, coconut coir, natural molasses, gypsum, and a fungus are the exception.

“Numerous disadvantages exist when using currently available media; rock wool products, although naturally derived, do not degrade, synthetic fiber or foam growing media [generally] consume petroleum in their manufacture and likewise do not degrade, finally, the peat based media are bound together with synthetic polymers. Pollutants, waste, and chemicals can leach into the soil from a synthetic-based medium. Synthetic growing medium is generally not biodegradable and contributes to solid waste at landfills.” [Id., amendment added.]

Jacob et al. teaches “a hydrophilic plant growth substrate, based on a coherent matrix of mineral wool which is hydrophilic due to the use of a specific cured resin . . . Although these types of known plant growth substrates are widely used, there is constant research to improve plant growth substrates, in particular the phytotoxicity of the chemicals used. This phytotoxicity may result . . . due to the binder.” [emphasis added.] (See U.S. Pat. No. 6,032,413 A, Pub. on Mar. 7, 2000, to Jacob et al.)

So impetuously widespread is the reliance on chemicals to bind or mix with soilless substrate particles, that one patent claims at least one “bases, including alkali hydroxides such as, for example, sodium hydroxide and potassium hydroxide, amines, sodium borohydrate, carboxymethyl acid, carboxymethyl chloride, surfactants, humectants, polyol molecules that interact with chitin and/or chitosan, glycerol, sorbitol, plasticization agents such as triglyceride plasticizers, oils such as linseed oil, linoleic acid, drying oils, ionic and/or nonionic glycols, alkyloxides, polyol monomers, oligomers, polymers, copolymers and networks, salts, ionic liquids, and combinations thereof” must first come in contact with the soilless fungus based biocomposite material “to increase the hydrophilicity of [said] biocomposite material,” “thereby providing an absorbent biocomposite material . . . [rendered appropriate] for use in vegetation propagation and preservation.” [emphasis added.] (See Araldi et al., U.S. Patent Nos: WO2015050626A1 & US20150038326 A1, Pub. on Apr. 9, and Feb. 5, 2015, respectively.)

To further confuse matters, Sungro Horticulture teaches that “'soilless' media are often fortified with lime and/or dolomitic lime, to control the acidity, and fertilizer packages. Many mixes contain nitrogen from various ammonium and nitrate salts and condensation products of aldehydes and urea; phosphorus from single or triple superphosphate, calcium phosphate, ammonium and potassium phosphates; potassium supplied from salts such as potassium nitrate, potassium phosphate and potassium chloride; calcium from lime products, calcium nitrate, or sulphate; magnesium from dolomitic lime, magnesium nitrate and sulfate; sulphur as the calcium, magnesium or ammonium salt; the transition metals, iron, copper, manganese and zinc, generally added as chelates, oxides or sulfates; boron as an oxide or complex salt with calcium or sodium; and molybdenum as one of the molybdate salts.” [emphasis added, int. quotations omitted.] (See U.S. Pat. No. 6,074,988 A, Pub. on Jun. 13, 2000, to King et al.)

“The twelve elements listed above plus chlorine are generally thought to be the only required mineral nutrients for plants. Much research has shown that another [periodic] element, silicon, is also a required element for certain plants and levels above the critical requirements provide significant benefits to many plants.” [amendment and emphasis added.] (Id.)

Chitin is an organically derived biopolymer. Chitosan is a derivative of chitin. Glucosamine(8) is a derivative of chitosan which is comprised of oligosaccharides whose fundamental units are monosaccharides. Like the periodic element silicon, chitin and its derivative organic molecules also have significant implications for agronomy. “Chitin has . . . been used in agriculture either as a protein complex (U.S. Pat. No. 4,536,207) or in combination with various microbes (U.S. Pat. Nos. 6,524,998 and 6,060,429) . . . Chitosan in combination with other components has been used in agricultural applications. See e.g. U.S. Pat. Nos. 6,649,566; 4,812,159; 6,407,040; 5,374,627 and 5,733,851.” [internal quotations omitted.] (See U.S. Patent No. WO 2012175739 A1, Pub. on Dec. 27, 2012, to Agrinos et al; for fn(8), see https://en.wikipedia.org/wiki/Glucosamine (Adopted in its entirety and fully set forth herein.)

“Chitin is a major component of fungal cell walls and serves as a molecular pattern for the recognition of potential pathogens in the innate immune systems of both plants and animals. In plants, chitin oligosaccharides have been known to induce various defense responses in a wide range of plant cells including both monocots and dicots.” (See Plant Cells Recognize Chitin Fragments For Defense Signaling Through A Plasma Membrane Receptor, Hanae Kaku et al., May 23, 2006.)

“The use of HYTa alone or in combination with chitin, chitosan, glucosamine and/or amino acids (1) provides nutrients and elements in the soil that increase crop yields by 25-55%, (2) reduces green house gas emissions, (3) increases the efficiency of mineral fertilizers (3) reduces the use of conventional fungicides and other pesticides, (4) increases the production of plant growth regulators, (5) improves soil structure, tilth, and water penetration and retention, (6) cleans up chemical residues and (7) shifts soil pH toward neutral pH.” (See U.S. Patent No. WO 2012175739 A1, supra.)

Chitin may be “deacetylated to produce chitosan.” (See U.S. Pat. No. 6,310,188 B1, Pub. on Oct. 30, 2001, to Mukherjee et al.) The process may be as simple as heating the chitin and introducing altered PH conditions. “It is believed that the heating followed by immediate quenching enhances the formation of chitin chains in an amorphous or a relaxed form, making them more susceptible to attack by acid or alkali . . .” (Id.) “Production of chitin and chitosan from fungal mycelium . . . are apparently more effective in inducing the plant immune response and are potentially more suitable for agricultural applications.” (See Production and Characterization of Fungal Chitin and Chitosan, Tao Wu, 2004, p. 5.)

“[F]ungi . . . cell walls can contain up to 40% [chitin] of the wall dry weight. The fungal mycelium is a complex network of filaments made of cells . . . Fungi which contain sufficient amounts of chitin can be selected and grown specifically for the extraction of chitin. A few patent and patent applications refer to fungal mycelium as a potential industrial source of chitin, for instance patents U.S. Pat. Nos. 4,960,413, 6,255,085, 4,195,175, 4,368,322, 4,806,474, 5,232,842, 6,333,399 . . . Most of these documents disclose methods for preparing chitosan or chitosan-glucan from fungal mycelium.” (See U.S. Pat. No. 7,556,946 B2, Pub. on Jul. 7, 2009, to Versali et al.)

Bayer et al. teach a type of heterogeneous fungal binder and filler for materials in general, but do not teach how to provide the proper biochemical composition and mechanical structure appropriate to a soilless plant cultivation substrate. (See U.S. Patent No. 20,080,145,577 A1, Pub. on Jun. 19, 2008, to Bayer et al.) The same is true for fungal based binders in related prior art. (See, for example, Patents US 20,110,268,980 A1, Pub. on Nov. 3, 2011, to Kalisz et al., 20,120,135,504 A1, Pub. on May 31, 2012, to Philip Ross.)

One inventor obtained a patent over the addition of specific strains of fungi to plant growth mediums and substrates “consisting of soil, sand, compost, peat, rice hulls, coir, cocopeat, soilless growing media containing organic and/or inorganic ingredients, artificial plant-growth substrates, polymer-based growth matrices, hydroponic nutrient and growth solutions, organic soil amendments, and mixtures thereof,” but does not teach the process of inoculating said mediums and substrates with a fungus to provide the proper biochemistry, composition, and mechanical structure appropriate to a soilless plant cultivation substrate. (See U.S. Patent No. US 20,120,028,799 A1, Feb. 2, 2012, to Martin et al.)

Prior art further teaches substrates that incorporate said 12 essential fertilizing macronutrients, micronutrients, and trace elements into their final loose or solid forms. (See U.S. Pat. No. 8,911,525 B1, supra.) Similar prior art teaches how to construct a soilless substrate based on a fibrous protein like keratin, and then how keratin degrading micro-organisms can release nutrient fertilizers like nitrogen into the substrate of its guest plant. (See U.S. Pat. No. 9,249,061 B2, Feb. 2, 2016, to Harman et al.) Alternative art focusses on the composition or structure of a substrate, without supplementing any essential nutrient, to enhance the guest plant's absorption of soluble nutrients through mechanical advantages like capillary dynamics. (See U.S. Pat. No. 4,949,503 A, Pub. on Aug. 21, 1990, to Chistiaan et al.)

“Understood by [capillary dynamics] is the ability of the product to distribute the water reserve uniformly throughout the product, or to redistribute it if the water reserve is limited. Good capillary dynamics are of great influence on cultivation yields because as a result of them the maximum rootage volume in the product is determined, possible fluctuations in the electrical conductivity remain restricted and a good water transport is assured in periods during which as a result of evaporation the plant extracts much water from the product.” (Id.)

Related prior art further teaches how to deter evaporation. “It would be an advance in the art to provide a mechanism whereby to automatically store within a soil, such as near a plant root, near a rootball of a plant, within a pot or indoor planter, or the like, a mechanism to absorb water, releasing it over time while resisting evaporation.” (See U.S. Patent No. US 20,110,289,841 A1, Pub. on Dec. 1, 2011, to Tommy K. Thrash.) Clearly, hydrophilicity and evaporation resistance both impact the greater overall property of capillary dynamics, not just absorption.

Evaporation and thermal resistance are inherently related to the physical property of “air-filled pore space (Bunt, 1988). Air-filled pores allow for drainage and gas exchange between the root environment and the outside atmosphere. Various materials have been used to provide for air-filled pore space in substrates, with one of the most common being perlite (Bunt, 1988).” (See Root Substrate pH et seq., supra, p. 644.)

Related prior art also teaches an aerobic environment which, in turn, deters anaerobic pathogens. “[A]n aerobic microbial biomass and viability supporting container . . . The aerobic microbial biomass is stored in a container which is characterized as being oxygen permeable to the extent of maintaining at least approximately 5.5 ppm oxygen in the aerobic microbial biomass . . . ” (See U.S. Pat. No. 7,833,777 B2, Pub. on Nov. 16, 2010, to Ingham et al.)

“Among the pathogenic microorganisms frequently detected in hydroponic cultures, those producing zoospores . . . , are particularly well adapted to these cultivation systems. As zoospores can swim, recycling facilitates rapid dissemination and subsequent root infection of the whole culture. Disease epidemics can occur, particularly in periods of stress, because of high temperatures and the low concentrations of dissolved oxygen in the nutrient solution. Highly pathogenic Pythium species . . . caused root rot and wilting.” [citations omitted.] (See fn(6), Vallance, et al., supra, p. 4, Infections by zoosporic oomycetes.)

The issue of increased dissolved oxygen content in the growing medium and/or in the nutrient solution has been tackled in many mechanical ways. For example, the Johnson Patent teaches continuous direct air injection into the bottom of an impervious water/nutrient reservoir, but does not inject that air/gas directly in the grow medium in which the plant's roots are anchored. (See U.S. Pat. No. 8,667,734, supra.) Another approach is to design piping that delivers water and air pressure up to the container holding the soil or soilless substrate in which the plant's roots are anchored. (See U.S. Patent No. WO 2,014,123,722 A1, Pub. on Aug. 14, 2014, to Mark Randell Prescott.)

Some prior art teaches the use of a saprophytic fungus to deliver agricultural benefits to the Earth's microsphere and biosphere in combination with the use of cardboard boxes, but does not teach how to use said fungus as a binder of organic fibers to formulate a soilless substrate that has the proper biochemistry, composition, and mechanical structure appropriate to all direct and indirect factors of a soilless plant cultivation substrate. (See U.S. Patent No. US 20,050,176,583 A1, Pub. on Aug. 11, 2005, to Paul Stamets.)

Fungi are classified by their feeding methods, and are thus generally limited to parasites, saprophytes, endophytes, and mycorrhizal. (See “Mycelium Running: How Mushrooms Can Help Save the World®,” 2005, at pp. 23-34, by Paul Stamets.) Most prior art teaches only that a mycorrhizal fungi may be used as an additive to a soil or soilless substrate. (See, for example, Mass multiplication of AMF using soilless substrates, Mycorrhiza News, Vol. 20, No. 1, April 2008, and U.S. Pat. No. 5,002,603 A, Pub. on Mar. 26, 1991, to Safir et al., US 20,080,064,598 A1, Pub. on Mar. 13, 2008, to Rougemont et al., U.S. Pat. No. 6,576,457 B1, Pub. on Jun. 10, 2003, to Sui-Sheng T. Hua.) Prior art does not teach that fungi cell walls may be leveraged in a biochemically, structurally, and mechanically suitable way that is appropriate to the propagation and maintenance of said host substrate's guest plants, and do so without a need for chemical modification prior to implementing the use of said substrate.

Mycorrhizae, endophytes, and parasites all generally require a living guest plant to develop therewith, therein, or thereon, respectively. Saprophytes are so named because they feed off of inanimate fibers. This invention leverages the molecular composition of the cell walls of saprophytic fungi, both as a heterogeneous binder of inanimate organic fibers, and concurrently leverages its non-essential-nutrient chitin based molecules to, inter alia, “enhance crop production, increase plant defensive processes, decrease the level of plant pathogens and reduce the amount of fertilizer used.” (See U.S. Patent No. WO 2012175739 A1, supra.)

The ability to resist compaction is also a desirable feature of a soilless substrate which has been disclosed in prior art. “[A] soil-less sand based root zone medium for the production of turf grass sod which has good percolation rates, good bulk density and resists compaction . . . ” (See U.S. Pat. No. 6,694,670 B1, Pub. on Feb. 24, 2004, to Michael A. Egan.) Compaction resistance in the design of a soilless substrate is also disclosed when “[a] circular web or mat is suitably formed of loosely felted coconut fibre and is then sprayed with a latex emulsion.” (See U.S. Pat. No. 3,958,365 A, Pub. on May 25, 1976, to Athol T. Proctor.)

“Coconut coir pith is a by-product of the coconut husk fiber processing industry. Coir is the name given to the fibrous material that constitutes the thick mesocarp (middle layer) of the coconut fruit (Cocos nucifera) . . . It has been recognized that coconut coir pith material provides an excellent growing medium for plants and it has been suggested that coconut coir pith can provide an effective alternative to previously standard growing media such as [non-renewable] peat moss.” [amendment added.] (See U.S. Pat. No. 8,024,890 B2, Pub. on Sep. 27, 2011, to Bertin et al.)

“Coconut coir pith has a high water holding capacity, ideal porosity, high cation exchange capacity and high stability . . . ” (Id.) Being an organic fiber that possesses an innate cation exchange capacity, coconut coir pith can absorb and adsorb water and soluble nutrients, whereas mineral based wools can only absorb water and soluble nutrients. Mineral based wool substrates do not electrically adsorb nutrient cations and anions.

In contrast to absorption and water holding capacity, rice hulls are another organic source of fibers that simulate the moisture drainage properties of perlite. Perlite originates from the volcanic mineral “obsidian,” and is thus not a renewable resource. “None of the differences between equivalent [para-boiled rice hulls] and perlite-containing substrates was high enough to be problematic with respect to crop production and all of the chemical parameters were within acceptable ranges for unused root substrates.” (See Root Substrate pH et seq., supra, p. 664.) Finally, prior art teaches how natural minerals such as gypsum and kalicinite have been used to obtain agronomical benefits. (See U.S. Pat. No. CA 2,449,870 A1, Pub. on Dec. 19, 2002, to Walter H. Runkis.)

Prior art has even determined that the final geometric shape of a substrate, not just its biochemical, structural, or mechanical composition, will impact its performance. “The level of conduciveness to the diseases caused by a given pathogenic agent might be determined by the nature (structure, composition) of the growth substrate of the crop. For instance, rockwool is more conducive to Pythium root rot and crown rot in cucumber culture than coir dust, pumice and perlite (van der Gaag and Wever, 2005). Temperature and oxygen concentration did not explain the differences between the media but the higher incidence of disease on rockwool was associated with a much greater water content than in the three others. When the height of the rockwool slabs was increased, the percentage of diseased plants decreased. These results indicated that water content plays a major role in the development of root and stem rot and that the type and height of substrate are important tools for decreasing yield losses.” (See fn(6), Vallance, et al., supra, Influence of the kind of substrate on microflora, p. 3.)

“In containerized substrate, AW (AWCont) depends also on container geometry, in particular on its height, which influences drainage: the taller the container, the more drainage and [due to the effects of gravity] the less capacity media will have to hold water. Water container capacity is defined as the amount of water retained in a containerized substrate system after drainage from saturation, but before evaporation.” [amendment added.] (See Fertigation and Substrate Management in Closed Soilless Culture, Pardossi A., Carmassi G., Diara C., Incrocci L., Maggini R., Massa D., August 2011, p. 13.)

“One characteristic consumers typically share is they have a limited amount of space available for growing food and ornamental plants . . . A successful product must accommodate a diversity of aesthetic requirements (e.g., visual, auditory, gustatory) and a wide range of reasons for growing (e.g., alternative plant varieties, alternative horticultural methods) . . . Previous attempts by others to design such a product have failed due to system expense, complexity or simplicity, aesthetics, flexibility (plants number/variety or horticultural practices), lack of system robustness, and/or amount of prior knowledge or care required by the user . . . Plants need [a suitable photonic light footprint], water, nutrients, oxygen, carbon dioxide, appropriate temperatures, and time in order to grow. This invention provides devices and methods for easily growing a wide variety of plants that are healthier and more nutritious than plants grown in soil. This invention provides a novel hydroponics system that is self-contained, useful for germination through harvest, useful for cuttings, is useful with low technology components, is useful for single plants through agricultural production, and provides more oxygen to the plant roots than other hydroponic systems.” [emphasis added.] (See U.S. Patent No. US 20,050,257,424 A1, Pub. on Nov. 24, 2005, to Bissonnette et al.)

### BRIEF SUMMARY OF THE INVENTION

As disclosed herein, after colonization of the pasteurized organic fibers by said fungus, no contact with any “substance” is required to produce a hydrophilic soilless substrate, hereinafter the “colonized substrate”. It is the object of this invention to disclose a substrate with superior hydrophilic properties(9) and capillary dynamics(10), made only of organically derived fibers, natural molasses(11), natural gypsum(12), and a fungus that has been deactivated (killed) by exposure to microwave radiation, hereinafter the “final substrate.”

See (9) U.S. Pat. No. 6,032,413 A, Pub. on Mar. 7, 2000, to Jacob et al., and (10) U.S. Pat. No. 4,949,503 A, Pub. on Aug. 21, 1990, to Chistiaan et al., supra, (11) https://en.wikipedia.org/wiki/Molasses, (12) http://www.eurogypsum.org/about-gypsum/what-is-gypsum. (The contents of footnotes (9) through (12), above, are adopted in their entirety as though fully set forth at length herein.)

A further object of this invention is to disclose the type of fungus and organic fibers, natural carbohydrates and minerals, and processes required to produce a colonized substrate which, after deactivation, possesses superior capillary dynamics for use in organic propagation and preservation of plants, wherein the elements and molecules comprising said substrate further bestow innate biochemical, structural, and mechanical benefits to the rhizosphere of its guest plants.

Yet a further object is to disclose that an organic adhesive for organic particles, together with a trace amount of natural carbohydrates and Earth minerals, and a related pasteurization and deactivation process can produce a final substrate: 1. Whose biochemistry, composition, and structure are free of any phytotoxicity to its guest plants and 2. Whose molecules may come in contact with the rhizosphere of its guest plant, resulting in agronomical benefits that are not an essential fertilizing macronutrient, micronutrient, or trace element, and, 3. That the whole of said final substrate can be produced without chemical modification necessary to imbue said final substrate with superior hydrophilicity and capillary dynamics.

On Feb. 23, 2016, this inventor publically disclosed, in a second provisional patent, a means of growing, as opposed to manufacturing, a fibrous grow substrate, by growing fungus on one or more pasteurized inanimate polymer fibers inside a corrugated and waxed cardboard box whose shape is a hexagonal prism. Said February 23rd provisional patent expressly references this inventor's first related provisional patent which discloses a means of increasing the dissolved oxygen of the nutrient solution through geometrically positioned biopolymer tubing comprising said substrates' entire silhouette and internal area, and thus its shape as a whole. (See provisional application Ser. No.: 62/192,567, filed Jul. 15, 2015, and Ser. No. 62/298,473, filed on Feb. 23, 2016, both from Hans Croteau.)

Yet a further object of this invention is to disclose that a hexagonal prism is the preferred shape because said shape imbues said final substrate with superior drainage properties and maximum use of floor space under any photonic light footprint. Said related choice of organic fungal inoculum, types of one or more organic biopolymer fibers used, said pasteurization process, said choice of natural carbohydrates and Earth minerals, and said chosen means of providing internal aeration, were all balanced to positively impact said final substrate's ability to drain, yet exhibit superior capillary dynamics.

It is yet a further object to disclose in general that optimal substrate performance requires exacting control over, inter alia, PH, PPM, Temperature, Biocompatibility, Density, Cationic Exchange Capacity, Electrical Conductivity, Percentage of Air Filled Pores, Permeability, and Absorbability. At least as is the case with live pleurotus ostreatus, said control is attenuated. The preferred embodiment is thus now to deactivate (kill) the colonized substrate by exposure to microwave(13) radiation which the FDA has declared generally safe. Microwaves do not render their subject molecules inorganic, nor do microwaves induce any chemical modification. In addition, the preferred composition of said colonized substrate is now 50% coco coir/pith, and 50% rice hull fibers; molasses and gypsum are added during pasteurization of said biopolymer fibers in preparation for colonization by the fungus (a.k.a., “mycological material”), thus forming the colonized substrate over a time period of approximately 3-5 days under a maintained atmosphere, barometric pressure, and a hygienically suitable environment. (For fn(13), see http://www.fda.gov/radiation-emittingproducts/resourcesforyouradiationemittingproductsucm252762.htm#Microwave_Oven_Safety_Standard; also see https://chriskresser.com/are-microwave-ovens-safe (Adopted in their entirety as though fully set forth herein.)

It is yet a further object of this patent to disclose that in preparation for commercial sale and distribution, a preferred embodiment now includes the placement of the final substrate that has been deactivated, in a sealed room with a dehumidifier set to its lowest setting, until said colonized substrate weighs approximately one half of its initial boxed weight. Said boxed weight is determined after colonization by the mycological material is complete, but before deactivation by microwave radiation, hereinafter referred to as the “final boxed substrate.”

It is the final object to disclose the best use and advantages of said final boxed substrates. Use of a tea brewed from the appropriate PH and temperature water together with beneficial micro-organisms is recommended to rehydrate said final boxed substrate before implementation and use as a soilless means of propagating and preserving plants. (See FIG. 5.)

It is yet a further object of this invention to disclose the results of this inventor's experimental analysis on said final boxed substrate, thus disclosing its identifiable genotypical and phenotypical advantages over the prior art. Under typical environmental conditions, proper use, and with a tomato plant as its guest, said final boxed substrate's internal fibrous surface was quickly colonized by beneficial fungi producing enzymes and chitinolytic bacteria, a.k.a., micro-biological activity, wherein said biology is capable of deacetylating chitin.

It is this inventor's opinion that said final boxed substrate increased the efficiency of mineral fertilizers, reduced the need for use of conventional fungicides and other pesticides, increased production of plant growth regulators, improved resistance to external structural manipulation, improved stability and control of its internal temperature, improved stability of its internal PH of water/nutrient solution for longer periods of time, improved capillary dynamics, improved water penetration and retention, improved resistance to evaporation, and that the resulting harvest had a higher yield, and was tastier and more robust than plants grown by any method or together with anything disclosed by all prior art.

### BRIEF DESCRIPTION OF THE DRAWINGS

FIG. 1 is a black and white schematic disclosing the cuts and creases (necessary to the related field of corrugation) to produce said preferred wax impregnated cardboard box;

FIG. 2 is a black and white drawing of a dotted view of said substrate through its box, disclosing all elements necessary during preferred use, including the functional air pump that said currently preferred (dotted) soaker hose based air/gas pressure diffuser is connected to;

FIG. 3 is a black and white drawing disclosing an alternate preferred embodiment of said air/gas pressure diffuser to deliver air, water, and/or nutrients incorporated through the wax impregnated flutes of one or more interconnected yet isolated final boxed substrates;

FIG. 4 is a black and white picture of said colonized substrate before deactivation by exposure to microwave radiation;

FIG. 5 is the black & white instructions printed on the exterior of said wax impregnated cardboard box's subsection marked “C”;

FIG. 6 is a black & white picture of said colonization drum.

### DETAILED DESCRIPTION OF THE INVENTION

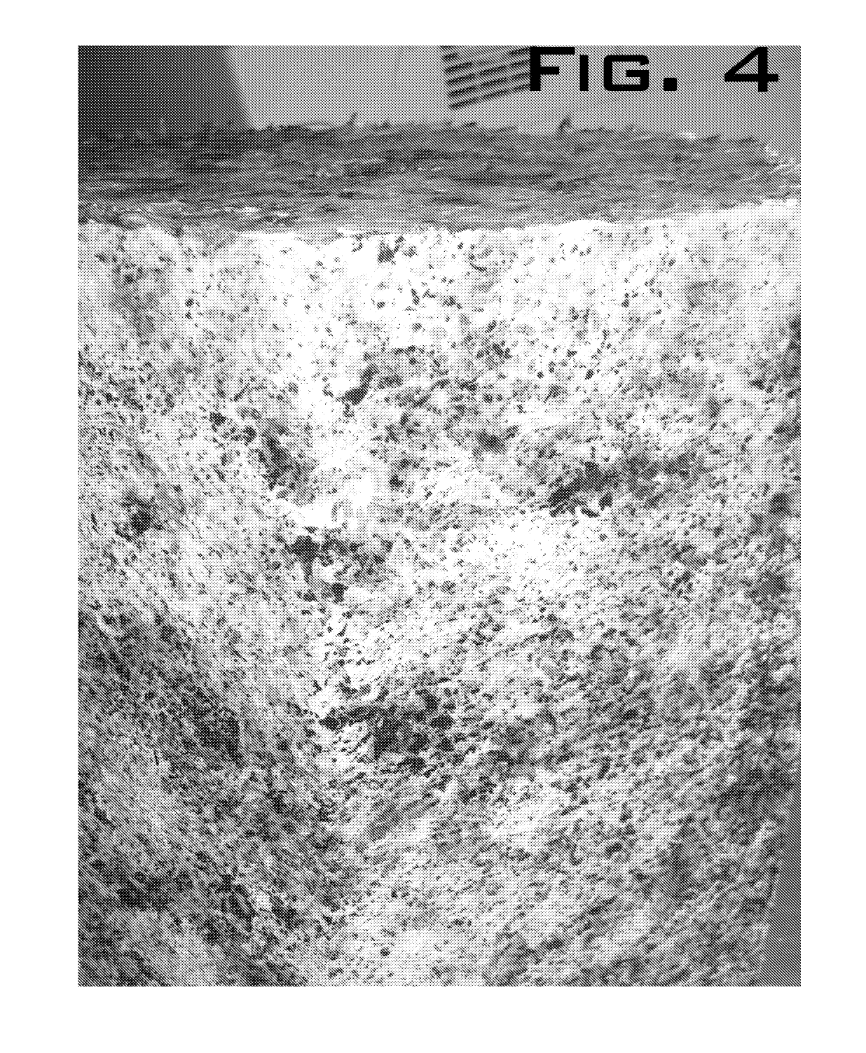

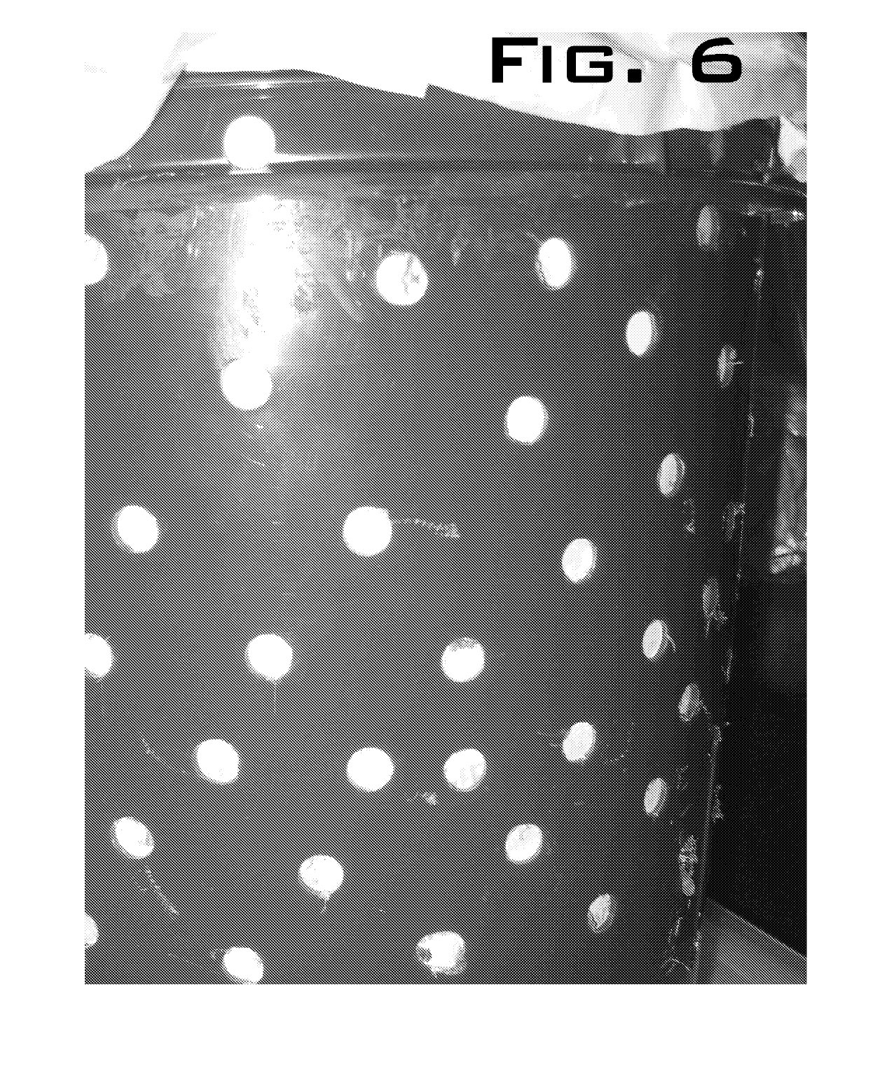

In a four-step process of making said invention, entailing: 1. Spawn Preparation, 2. Pasteurization, 3. Colonization, and 4. Deactivation, any type of fungus may be grown on any type of polymer that has been prepared alone, or with any type of organic matter, or natural Earth minerals, or combination of said former two, with said resulting biocomposite mixture then being placed in any type and shape of container and provided appropriate time, atmosphere, barometric pressure, and hygienic conditions for said fungus to colonize said polymers over time, thus forming said non-compacting, light weight, non-vitreous, rigid foam structure hereinabove referenced as said colonized substrate (i.e., see FIG. 4), but the preferred fungus is pleurotus ostreatus which has been “spawned” off of sterilized whole corn kernels in a 5 gallon bucket which has been perforated with approximately 250 evenly spaced ½ inch diameter holes, with the interior of said bucket being lined with densely interwoven high-density polyethylene fibers, such as Tyvek(14), acting as a filter, the whole of which is hereinafter referred to as the “colonization drum.” (See FIG. 6; fn(14), Tyvek® is a registered trademark of the Dupont™ Corporation, and is flashspun high-density polyethylene fibers.)

Fungi include, but are not limited to, spores, mycelium, inoculum of any type of fungus, or any polymer, solute, or solution having come in contact with any fungus. Sterilization of said whole corn kernels is accomplished by placing said whole corn kernels in any heated pressure retaining enclosure, such as a pressure cooker, together with an amount of water that is equal to the volume of said unsaturated corn kernels, with said water containing 0.12% gypsum of the total weight of said unsaturated whole corn kernels, and with said mixture then heated sufficiently to reach 15 pounds per square inch of internal pressure for 2 hours.

After sterilization, said whole corn kernels are cooled down to between 77 and 85 degrees Fahrenheit in a hygienically appropriate atmosphere before being placed inside said colonization drum along with any fungi, wherein said spawn is then kept in a HEPA filtered oxygen rich environment for approximately three weeks, hereinafter referred to as Spawn Preparation.

In the second step of said four-step process, any type of one or more polymers may be prepared with any type of organic matter, or natural Earth minerals, or combination with organic matter and natural Earth minerals, wherein said prepared polymers thereafter come in contact with said previously prepared spawn, forming a biocomposite mixture, wherein said biocomposite mixture is then placed in any type and shape of container and provided appropriate time, atmosphere, barometric pressure, and hygienic conditions for said spawn to colonize said polymers, thus forming said colonized substrate (i.e., see FIG. 4), but the preferred embodiment is a mixture of 50% coco coir pith (with a measurable electrical soluble conductivity of no more than 0.2 units, according to Hana Instruments' scale), and 50% rice hulls, wherein said polymer mixture is pasteurized for two hours at 150 deg. Fahrenheit in any container in dechlorinated water of volume equal to said volume of unsaturated coco coir pith and rice hulls, and wherein said water contains 0.5% in molasses and 0.12% in gypsum of the total volume of said unsaturated coco coir pith and rice hulls.

A sufficient amount of polymers necessary to loosely fill said waxed corrugated box must be pasteurized in preparation for contact with said spawn, which, in order to make a final boxed substrate with a 10 inch height and a “pie calculation” of 3.2 (explained infra), approximately 5.5 pounds of said dry polymer weight is needed. After pasteurization, said polymers must be drained to remove all standing water, must have a moisture content of approximately 60%, and must be cooled down to between 77 and 85 deg. Fahrenheit in a hygienically appropriate atmosphere, wherein said polymers then contact approximately 56 ounces of said previously prepared spawn, forming an inoculated biocomposite mixture.

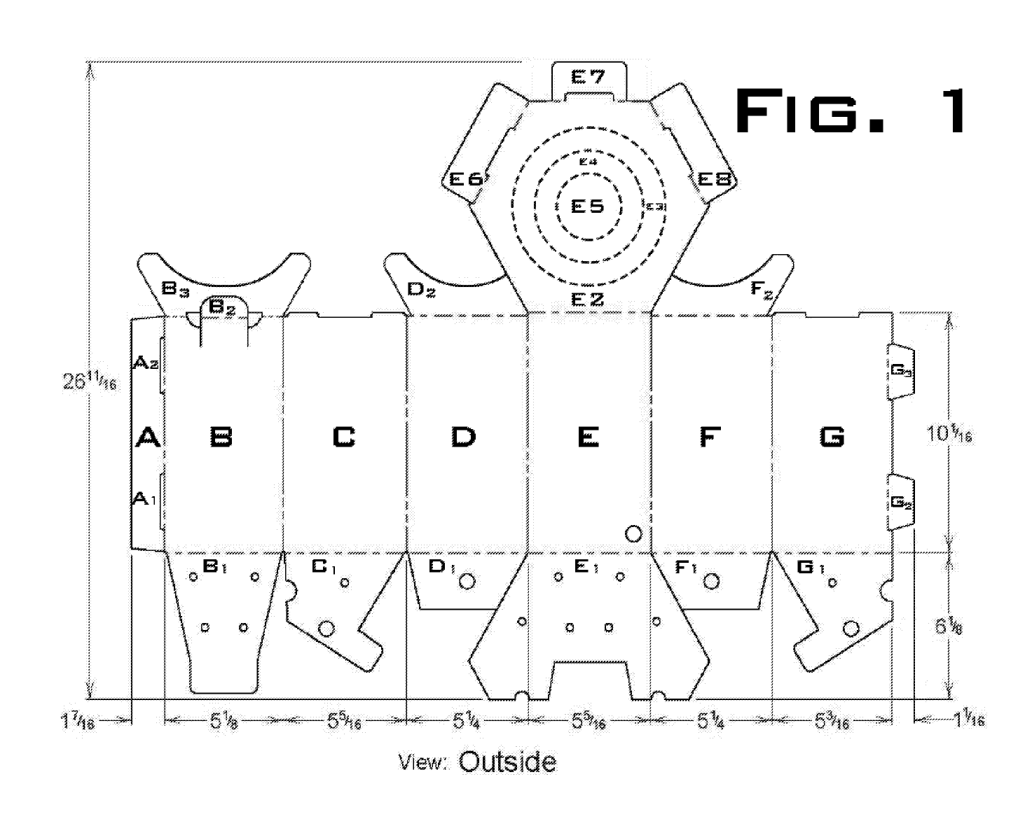

In the third step of said four-step process, said inoculated biocomposite mixture is placed in any type and shape of container with an appropriately high gas exchange barrier permitting respiration(15), wherein said biocomposite mixture is kept in a hygienically appropriate atmosphere, barometric pressure, temperature, and low lumen intensity for approximately 3-5 days, but the preferred container is a hexagonally shaped and externally printed fiber paper corrugated box, commonly referred to as “cardboard,” wherein said cardboard has been dipped in mineral wax, also known as “cascade waxed,” and wherein said cardboard box has a “hexagonal pie calculation” of 3.2 units. (See FIG. 1.) Said “hexagonal pie calculation” is a measurement of the total units of circumference of the hexagonal prism shape of the colonized substrate, divided by its total height. So, for example, a 32 inch hexagonal circumference with a 10 inch height has a “hexagonal pie calculation” of 3.2 unit inches. (For fn(15), see http://hyperphysics.phy-astr.gsu.edu/hbase/biology/respir.html, whose contents is incorporated herein as though fully set forth at length.)

Said preferred waxed corrugated box must be prepared ahead of the inoculation process, must contain drain holes at its bottom, and must accommodate an access hole on one edge of said box to accommodate said air pressure diffuser connector. The preferred embodiment incorporates said waxed corrugated box cover as part of the whole box. (Id.) Preferably, said waxed corrugated box is made of paper pulp that has been pasteurized.

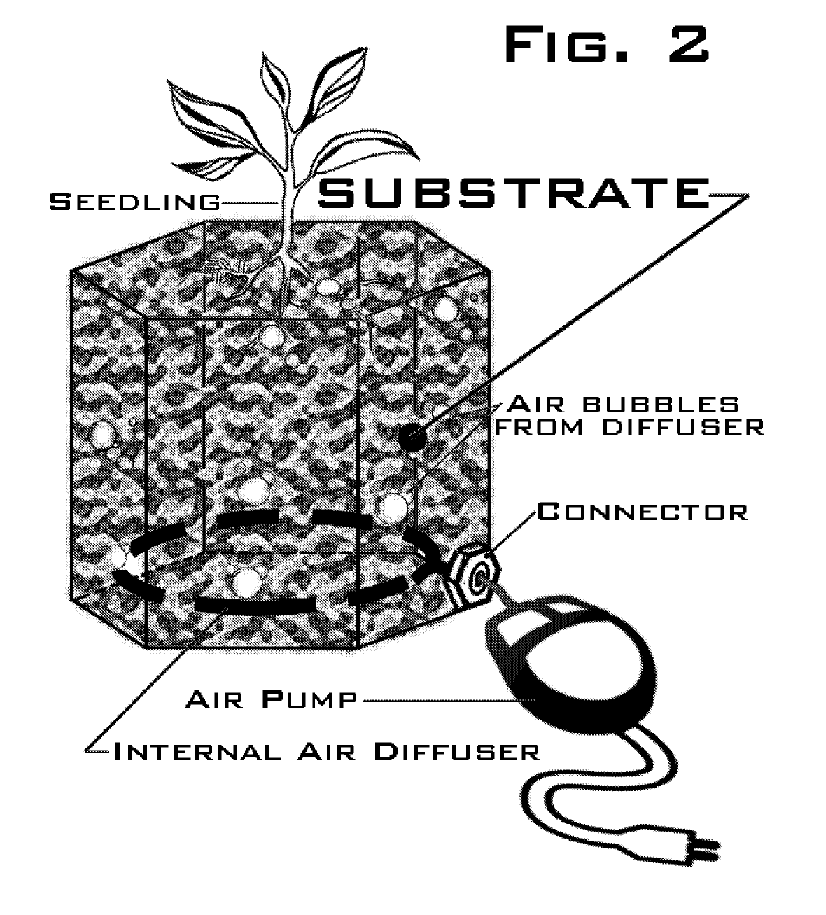

Any air/gas pressure diffuser made of any material, including but not limited to, an air pressure diffuser composed of a corn starch base or similar organic matter which biodegrades only under thermophilic activity, may be located anywhere inside said inoculated biocomposite mixture and final substrate, but the current preferred composition of said air/gas pressure diffuser is sintered recycled tire rubber, also known as a “soaker hose,” is connected to an interior to exterior accessibility accommodation located approximately ½ inch above the bottom corner of said final boxed substrate on its subsection “E”. (See FIG. 2.)

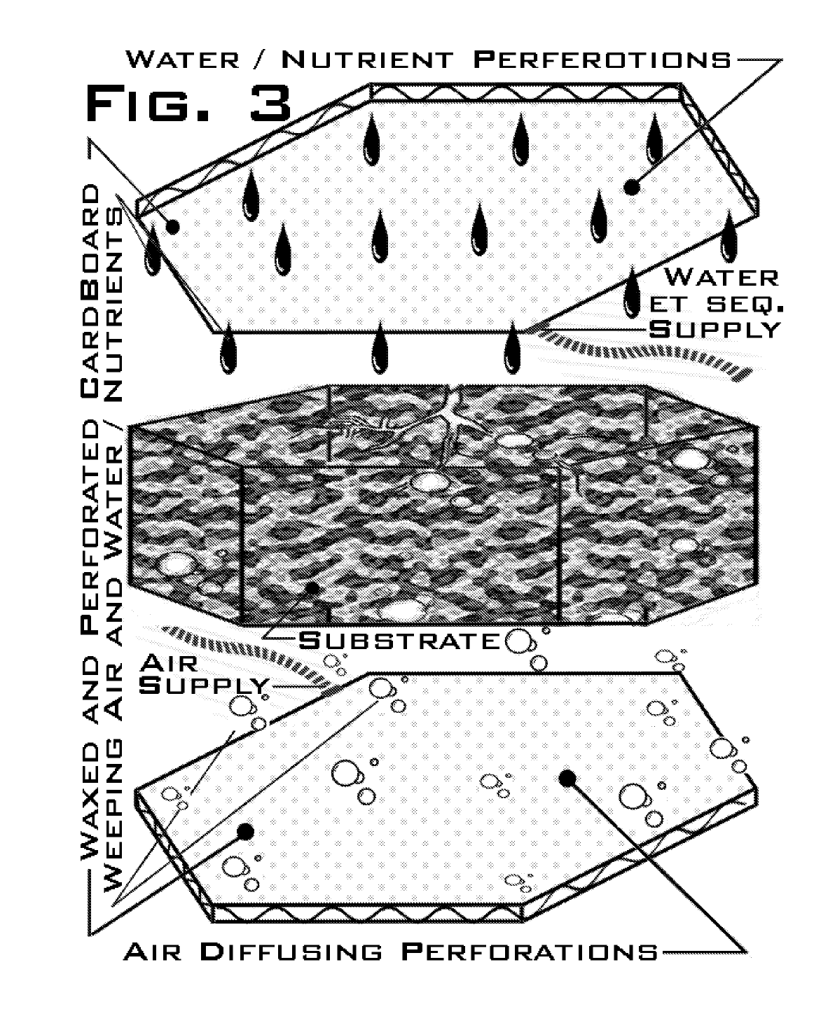

An alternate preferred embodiment of an air/gas pressure diffuser is to deliver air, water, and/or nutrients under pressure through the wax impregnated flutes of one or more interconnected final boxed substrates, wherein said air, water, and/or nutrients each remain isolated from the remaining interconnected final boxed substrates. Said alternate preferred method requires that perforations be made to the inner liner of the top and bottom of said waxed corrugated box. With gravity, said perforations in the box's top section permit the water and nutrient to drain down atop of said substrate, while the perforations in its bottom section permit the air and gas to rise through and internally aerate said substrate. (FIG. 3.)

In the fourth step of said four-step process, said final boxed substrate may be deactivated in numerous ways, including but not limited to, being placed in a complete vacuum for a sufficient period of time, being boiled for a sufficient period of time, or being exposed to conventional heat for a sufficient period of time, but for the sake of preserving energy and economy, the preferred fungal deactivation means is to expose said colonized substrate to microwave radiation for approximately 1 min per every inch of height of said colonized substrate's embodied measurement.

In preparation for commercial distribution, and in order to reduce its weight, said final boxed substrate is then placed in a clean room that is equipped with a functioning and filtered dehumidifier until said final boxed substrate weighs approximately one half of what said colonized substrate weighed before deactivation by microwave radiation.

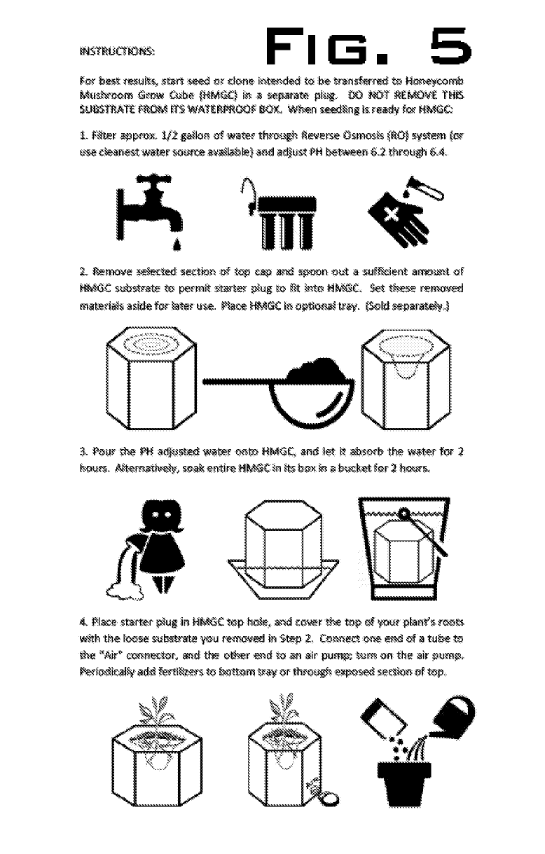

In a four-step process of using said final boxed substrate, entailing: 1. Filtered Water Preparation, 2. Spooning Out Extra Foam, 3. Rehydration, and 4. Transplantation or Germination, any type of plant may be grown in or on said final boxed substrate by contacting any part of said final boxed substrate with any part of plant matter (propagation), but the preferred use is to start a seed or clone, intended to be transferred to said final boxed substrate, in a separate “starter plug.” (See FIG. 5.) It is not recommended to remove said colonized and deactivated biocomposite substrate from its waterproof wax impregnated box.

When said “starter plug” or plant seed is ready to be rooted inside said final boxed substrate, any type of moisture may be chosen, including but not limited to, tap water, but the preferred use comprises of filtering approximately ½ gallon of water through a Reverse Osmosis (RO) system, or at least a through a filter capable or removing chlorine, and then adjusting said filtered water's PH between 6.2 through 6.4, hereinafter referred to as said Filtered Water Preparation.

In Step 2, Spooning Out Extra Foam, any pre-perforated cardboard section of said boxed final substrate that is large enough to permit a sufficient amount of underlying biocomposite material to be removed, thus permitting said seed or “starter plug” to fit inside said final boxed substrate, may be removed prior to or after rehydration, if any at all, but the preferred use is to select and remove one of three said top pre-perforated cardboard sections of sufficient size to permit the removal of enough underlying biocomposite material and permit said “starter plug” to fit inside said biocomposite material, deep enough to permit its guest plant's roots to be covered with an even layer of the loose biocomposite material which may have been previously removed and set aside for later use. Said final boxed substrate may also be placed in an optional and impermeable tray.

In Step 3, Rehydration, said final boxed substrate may come in contact with any type of moisture at any temperature or PH, and which contains any type or combination of PPMs, organic matter, or microorganisms, but the preferred use is to mix any amount of fresh compost tea(16) with said PH adjusted water, at room temperature, and then pour said resulting solution onto the exposed top surface of said final boxed substrate, and then allow said substrate to absorb said solution for 2 hours. Alternatively, said final boxed substrate may be submerged in its waterproof box in a bucket containing said solution for approximately 2 hours. (For fn(16), see U.S. Pat. No. 7,833,777 B2, supra.)

In Step 4, Transplantation or Germination, any plant matter may come in contact with any part of said rehydrated final boxed substrate, and then maintained under any photonic light footprint at any temperature and under any atmospheric conditions, but the preferred use is to place said starter plug in said hole created by the previous removal of extra biocomposite material, if any, and to cover the top of said guest plant's roots with said loose biocomposite substrate material removed in Step 2, if any. (Id.)

An alternative preferred use, when no loose substrate has previously been removed, is to place said seed inside a small incision made atop of said final boxed substrate's exposed surface. For best performance, an impermeable tube should be connected to the push in connector of the final boxed substrate's “E” subsection marked “Air”, wherein the other end of said impermeable tube is connected to a functioning air pump. (See FIG. 2.) For best performance of said final boxed substrate, water and fertilizers should be added periodically to the bottom tray or through the exposed section of said final boxed substrate's exposed top surface, the same way a typical potted plant would be watered and fertilized.

1. A micro-biologically favoring fungus based biocomposite substrate having superior capillary dynamics comprising:
a. one or more Fungi, and,
b. one or more polymers, alone, or prepared with organic matter or natural minerals, or prepared with a combination of organic matter and natural minerals.

2. The biocomposite substrate of claim 1, further comprising a means of internal aeration.

3. Said means of internal aeration of claim 2, wherein said means of internal aeration is provided by a porous hose made of sintered rubber particles.

4. Said means of internal aeration of claim 2, wherein said means of internal aeration is provided by a porous hose made of materials that biodegrade under thermophilic activity.

5. The biocomposite substrate of claim 1, wherein said biocomposite substrate is mostly encapsulated by a container.

6. The biocomposite substrate of claim 1, wherein said one or more polymers is comprised of approximately 50% coco coir pith and approximately 50% rice hulls.

7. The biocomposite substrate of claim 1, wherein said organic matter includes approximately 0.5% in molasses of the total volume of said one or more polymers.

8. The biocomposite substrate of claim 1, wherein said natural minerals include approximately 0.12% in gypsum of the total volume of said one or more polymers.

9. The biocomposite substrate of claim 1, wherein said one or more polymers has been pasteurized alone, pasteurized with organic matter or natural minerals, or pasteurized with a combination of organic matter and natural minerals.

10. A method of making a micro-biologically favoring fungus based biocomposite substrate having superior capillary dynamics comprising:
a. preparing one or more fungi,
b. preparing one or more polymers, alone, or with organic matter or natural minerals, or with a combination of organic matter and natural minerals,
c. contacting said prepared polymers with said one or more prepared fungi resulting in a biocomposite,
d. placing said biocomposite in a container, and,
e. maintaining the temperature of said container for some time.

11. The method of claim 10, wherein preparing one or more fungi involves mixing said one or more fungi with sterilized and cooled organic matter, resulting in spawn.

12. The method of claim 11, wherein said spawn preparation involves encapsulation of said spawn by interwoven polymer fibers.

13. The method of claim 12, wherein said spawn encapsulated by said interwoven polymer fibers is thermally maintained for approximately three weeks.

14. The method of claim 10, wherein preparing said one or more polymers involves pasteurization of said one or more polymers alone, pasteurization of said one or more polymers with organic matter or natural minerals, or pasteurization of said one or more polymers with a combination of organic matter and natural minerals.

15. The method of claim 10, wherein said one or more fungi comprising said biocomposite comprising said container is thereafter deactivated.

16. The method of claim 10, wherein said container is any shape box.

17. Said box of claim 16, wherein said box has a “hexagonal pie calculation” of approximately 3.2 units of any measure.

18. Said box of claim 16, further comprising wax impregnation.

19. Said box of claim 18, further comprising an incorporated means of delivering air, water, or nutrients.

20. The method of claim 15, wherein said deactivated container is thereafter dehydrated down to approximately ½ of said deactivated container's initial weight.
